(19) United States
(12) Patent Application Publication
(10) Pub. No.: US 2025/0057606 A1
(43) Pub. Date: Feb. 20, 2025

(54) APPARATUS FOR ROBOT-ASSISTED ELECTROSURGERY

(71) Applicant: Creo Medical Limited, Chepstow, South Wales (GB)

(72) Inventors: Christopher Paul Hancock, Chepstow, South Wales (GB); John Bishop, Chepstow (GB)

(73) Assignee: Creo Medical Limited, Chepstow, South Wales (GB)

(21) Appl. No.: 18/580,595

(22) PCT Filed: Jun. 9, 2022
(86) PCT No.: PCT/EP2022/065731

§ 371 Date: Jan. 18, 2024

(30) Foreign Application Priority Data
Jul. 30, 2021 (GB) 2111041.6

(51) Int. Cl. A61B 34/30; A61B 18/02; A61B 18/12; A61B 18/18

(52) U.S. Cl. CPC A61B 34/30; A61B 18/02; A61B 18/1206; A61B 2018/1823

(57) ABSTRACT

Various embodiments provide an apparatus for a robot-assisted surgical system. The apparatus comprises a robotic surgical tool comprising an articulated robot arm for supporting an electrosurgical instrument. The apparatus also comprises a cooling assembly for removing heat from an electrosurgical generator unit mounted to the robotic surgical tool, the electrosurgical generator unit for generating an electrosurgical signal for use by the electrosurgical instrument.

This application claims priority from GB2012303.0 filed 7 Aug. 2020, the contents and elements of which are herein incorporated by reference for all purposes.

### FIELD OF THE INVENTION

The invention relates to an apparatus for a robot-assisted surgical system. In particular, the invention relates to various cooling arrangements that can be incorporated into a robot-assisted surgical system to remove heat generated by an electrosurgical generator unit mountable to a robotic surgical tool.

### BACKGROUND

Electromagnetic (EM) energy, and in particular microwave and radiofrequency (RF) energy, has been found to be useful in electrosurgical operations, for its ability to cut, coagulate, and ablate body tissue. Typically, apparatus for delivering EM energy to body tissue includes a generator comprising a source of EM energy, and an electrosurgical instrument connected to the generator, for delivering the energy to tissue.

Tissue ablation using microwave EM energy is based on the fact that biological tissue is largely composed of water. Human soft organ tissue is typically between 70% and 80% water content. Water molecules have a permanent electric dipole moment, meaning that a charge imbalance exists across the molecule. This charge imbalance causes the molecules to move in response to the forces generated by application of a time varying electric field as the molecules rotate to align their electric dipole moment with the polarity of the applied field. At microwave frequencies, rapid molecular oscillations result in frictional heating and consequential dissipation of the field energy in the form of heat. This is known as dielectric heating.

This principle is harnessed in microwave ablation therapies, where water molecules in target tissue are rapidly heated by application of a localised electromagnetic field at microwave frequencies, resulting in tissue coagulation and cell death. It is known to use microwave emitting probes to treat various conditions in the lungs and other organs. For example, in the lungs, microwave radiation can be used to treat asthma and ablate tumours or lesions.

Surgical resection is a means of removing sections of organs from within the human or animal body. Such organs may be highly vascular. When tissue is cut (divided or transected) small blood vessels called arterioles are damaged or ruptured. Initial bleeding is followed by a coagulation cascade where the blood is turned into a clot in an attempt to plug the bleeding point. During an operation, it is desirable for a patient to lose as little blood as possible, so various devices have been developed in an attempt to provide blood free cutting.

Instead of a sharp blade, it is known to use radiofrequency (RF) energy to cut biological tissue. The method of cutting using RF energy operates using the principle that as an electric current passes through a tissue matrix (aided by the ionic contents of the cells and the intercellular electrolytes), the impedance to the flow of electrons across the tissue generates heat. When an RF voltage is applied to the tissue matrix, enough heat is generated within the cells to vaporise the water content of the tissue. As a result of this increasing desiccation, particularly adjacent to the RF emitting region of the instrument (referred to herein as an RF blade) which has the highest current density of the entire current path through tissue, the tissue adjacent to the cut pole of the RF blade loses direct contact with the blade. The applied voltage then appears almost entirely across this void which ionises as a result, forming a plasma, which has a very high volume resistivity compared to tissue. This differentiation is important as it focusses the applied energy to the plasma that completed the electrical circuit between the cut pole of the RF blade and the tissue. Any volatile material entering the plasma slowly enough is vaporised and the perception is therefore of a tissue dissecting plasma.

The use of robotic equipment to assist surgery is increasing rapidly. Typically robot-assisted surgery involves the use of a robotic arm, which can be controlled directly or remotely by a surgeon to perform various movements or manipulations of a given surgical procedure. The robotic arm may have an end-effector at a distal end thereof. The end-effector may be or may carry a surgical instrument. Robotic-assisted surgical systems may be used in open and laparoscopic procedures.

It is known to use robot-assisted surgical systems in electrosurgical procedures. For example, the Da Vinci™ system manufactured by Intuitive Surgical™ allows for a generator to be integrated into a vision cart that is connectable to the patient cart that carries the robotic arms.

The present invention has been devised in light of the above considerations.

### SUMMARY OF THE INVENTION

The present disclosure describes developments of a concept put forward in the applicant's earlier GB patent application no. 2012303.0, filed on 7 Aug. 2020, which is incorporated herein by reference. GB patent application no. 2012303.0 provides a robot-assisted surgical system in which an electrosurgical generator unit for providing electrosurgical functionality is directly mountable on or integrated within a robotic arm. The electrosurgical generator unit may be a detachable module (referred to herein as a “capsule”), which may be movable between different robotic arms in the same environment. The electrosurgical generator unit may comprise a plurality of modules, each providing a different treatment modality. Depending on the procedure to be performed, a different module or combination of modules may be selected and mounted on one or more robotic arms.

This arrangement may provide a number of advantages. Firstly, by mounting the electrosurgical generator unit directly on the robotic arm, the means for generating energy for electrosurgery can be brought closer to the electrosurgical instrument. This facilitates the reduction or elimination of losses that can occur in conveying energy between a generator and the electrosurgical instrument. Secondly, providing the electrosurgical generator unit on the robotic arm avoids the need for a separate piece of operating suite furniture to house the electrosurgical generator. This may save space in the operating theatre. Thirdly, providing a modular set-up may enable each robotic arm in a multi-arm system to have the same functionality without the cost of independently configuring each arm for electrosurgery.

The present disclosure further develops this concept. At its most general, the present invention provides a cooling assembly (also referred to herein as a cooling arrangement) to dissipate heat produced by the electrosurgical generator unit which is mountable to a robotic surgical tool.

When the electrosurgical generator unit is mountable on the robotic surgical tool (rather than e.g. in a separate piece of operating piece furniture), there may be a risk that heating of the electrosurgical generator unit could cause damage to the robotic surgical tool and/or to the electrosurgical generator unit itself. Additionally, since the generator unit is mountable to the robotic surgical tool (which is relatively near to the patient), it is preferable to keep safe temperatures in this area so as not to cause injury to a user (e.g. the patient or operator of the system). Further, since the electrosurgical generator unit is mountable on the robotic surgical tool, this may impose restrictions on a form factor (e.g. size, shape, etc.) of the mountable generator unit. For example, the mountable generator unit may have to be smaller than if it were not mountable on the robotic surgical tool so that the generator unit does not impede the operation (e.g. movement) of the robotic surgical tool (e.g. a robot arm). Therefore, owing to this reduced form factor, an amount of heat produced by the mountable generator unit per unit volume may be higher than an equivalent amount of heat produced by a non-mountable generator unit. As a result, there may be an increased need to cool a mountable generator unit compared to, for example, a non-mountable generator unit.

By providing a cooling arrangement for dissipating heat from the electrosurgical generator unit, the present invention can mitigate these risks whilst also providing the above advantages associated with the priority application. That is, the present invention helps to provide an arrangement in which a generator can be provided directly on a robotic surgical tool, whilst also ensuring reliable performance of the robotic surgical system.

According to a first aspect of the invention, there is provided an apparatus for a robot-assisted surgical system, the apparatus comprising: a robotic surgical tool comprising an articulated robot arm for supporting an electrosurgical instrument; and a cooling assembly for removing heat from an electrosurgical generator unit mounted to the robotic surgical tool, the electrosurgical generator unit for generating an electrosurgical signal for use by the electrosurgical instrument.

For example, the cooling assembly may include one or more passive cooling arrangements and/or active cooling mechanisms for removing heat from the electrosurgical generator unit when mounted to the robotic surgical tool. As used herein, an “active cooling mechanism” refers to a cooling mechanism which requires power (e.g. electrical power) to perform cooling. A “passive cooling mechanism” refers to a cooling arrangement which does not require power, e.g. by selecting the shape and/or materials of the apparatus to promote heat dissipation. Any combination of active and/or passive cooling mechanisms may be provided to improve heat dissipation.

The robotic surgical tool may also be referred to as a robotic surgical unit, a robotic surgical apparatus, or robotic surgical device. The robotic surgical tool may further include a control column (also referred to as a support structure or plinth) to support the articulated robot arm.

The cooling assembly may be provided on any part of the apparatus (e.g. as part of the robotic surgical tool such as the articulated robot arm or the support structure for the arm, as part of the electrosurgical generator unit, or as a separate component).

The cooling assembly is arranged for thermal communication with (i.e. is thermally coupled to) the electrosurgical generator unit when mounted to the robotic surgical tool. That is, the apparatus includes a heat flow path for thermally coupling the cooling assembly to the electrosurgical generator unit when the electrosurgical generator unit is mounted on the robotic surgical tool. The heat flow path may include one or more physical elements (e.g. cooling mechanisms such as thermally conductive elements) which are physically connected together (e.g. in a chain) and which physically contact to the electrosurgical generator unit and to the cooling assembly. Alternatively, the heat flow path may include one or more gaps between the electrosurgical generator unit and the cooling assembly (e.g. a gap between two adjacent elements in the chain), where the gap is dimensioned to permit thermal communication between the electrosurgical generator unit and the cooling assembly. For example, the gap may be substantially narrow so as to permit heat flow from the electrosurgical generator unit toward the cooling assembly. Alternatively or in combination, the cooling assembly may comprise a fan, and the gap may permit airflow from the fan toward an element of the apparatus (e.g. directly onto the electrosurgical generator unit, onto another element such as the robotic surgical tool or another cooling mechanism).

Therefore, as is described further herein, the cooling assembly may be arranged for direct thermal communication (i.e. by providing direct physical contact) or indirect thermal communication (by permitting a physical gap) with the electrosurgical generator unit (once mounted) for dissipating heat produced by the electrosurgical generator unit.

The cooling assembly may be configured to maintain a low temperature, so as to provide a thermal gradient (e.g. across the heat flow path) that promotes directional heat flow from the electrosurgical generator unit towards cooling assembly when the electrosurgical generator unit is at high temperatures. For example, this may be achieved by including an active cooling mechanism (e.g. a fan) for actively providing a low temperature at the cooling assembly, and/or by including a passive cooling mechanism (e.g. fins or a heat sink) to promote fast heat dissipation from the cooling assembly.

Similarly to the electrosurgical generator unit, the cooling assembly may be detachably mountable to the apparatus, which may permit it to be exchanged between different apparatus within the same operating theatre environment. The cooling assembly may be capable of retrofitting to existing apparatus.

The apparatus may further comprise a connector for mounting the electrosurgical generator unit to the robotic surgical tool.

The connector may be provided on any part of the apparatus (e.g. as part of the robotic surgical tool such as the articulated robot arm or a support structure for the arm, or as part of the electrosurgical generator unit), or as a separate component (e.g. straps). The connector may be any suitable element that forms part of a connection between the electrosurgical generator unit and robotic surgical tool. For example, the connector may include a socket or recess (e.g. in the robot arm and/or control column) for receiving the electrosurgical generator unit, or may include snap-fit elements or a separate strap for securing the electrosurgical generator unit to the robotic surgical tool.

The apparatus may further comprise the electrosurgical generator unit, said electrosurgical generator unit comprising: a housing; a signal generator contained within the housing, the signal generator being configured to generate the electrosurgical signal for use by the electrosurgical instrument; and an energy delivery structure to couple the electrosurgical signal into the robotic surgical tool.

The electrosurgical generator unit may be configured to generate various types of electromagnetic energy for use in the electrosurgical instrument. For example, radiofrequency and/or microwave energy may be generated to treat or measure biological tissue. For example, radiofrequency and/or microwave energy may be used to perform any of ablation, haemostasis (i.e. sealing broken blood vessels by promoting blood coagulation), cutting, sterilization, etc.

The energy delivery structure serves to couple the electrosurgical signal into the robotic surgical tool for use by the electrosurgical instrument. For example, the energy delivery structure may form at least part of a feed structure which extends between the signal generator and the instrument, via the robotic surgical tool (e.g. articulated robot arm and/or control column) so that the instrument is powered by the signal generated by the signal generator. That is, the energy delivery structure may include a part of the feed structure which is located within the housing of the generator unit. In an embodiment, the feed structure may travel over an outer surface of the robotic surgical tool and/or inside an outer housing of the robotic surgical tool. In an embodiment, the feed structure may include a cable or cable assembly, for example, including at least one coaxial cable.

The electrosurgical generator unit may be detachably mountable to any portion of the robotic surgical tool, e.g. to the articulated robot arm or to a control column of the robotic surgical tool. For example, the housing of the electrosurgical generator unit may be secured to the robotic surgical tool via a separate connector (e.g. straps), or via an integrated connector (e.g. snap-fit arrangement). The integrated connector may be incorporated in the structure of the robotic surgical tool and/or electrosurgical generator unit for connecting the electrosurgical generator unit to the robotic surgical tool. That is, as mentioned above, the connector for mounting the electrosurgical generator unit to the robotic surgical tool may be part of the generator unit and/or the robotic surgical tool.

The electrosurgical generator unit may be provided on the same or different part of the apparatus from the cooling assembly. For example, the cooling assembly and/or electrosurgical generator unit may both be provided on a control column of the robotic surgical tool (e.g. at an opposite end of the articulated robot arm from the end which supports the electrosurgical instrument). This may help to dissipate the heat produced by the electrosurgical generator unit further from the patient, e.g. compared to an electrosurgical generator unit and/or cooling assembly on the articulated robot arm at or near to the electrosurgical instrument.

Further details regarding possible configurations of the electrosurgical generator unit are provided in the priority application, GB patent application no. 2012303.0, which is incorporated herein by reference.

The cooling assembly may comprise a heat sink arranged to be in thermal communication with the electrosurgical generator unit when the electrosurgical generator unit is mounted to the robotic surgical tool.

Preferably, the heat sink is formed of a thermal conductive material. For example, the heat sink may be formed of a metal such as copper, aluminium, or brass. This may allow the heat sink to conduct energy away from the electrosurgical generator unit, allowing heat to preferentially flow from the electrosurgical generator unit to the heat sink.

The heat sink may have a relatively large heat capacity, e.g. greater than the heat capacity of the electrosurgical generator unit (e.g. greater than a heat capacity of the housing of the electrosurgical generator unit). This may help the heat sink to absorb excess heat, with a minimal corresponding increase in temperature at the heat sink.

The heat sink may be a separate structure from the robotic surgical tool and/or electrosurgical generator unit (e.g. a separate piece of metal). Alternatively, the heat sink may be incorporated into the apparatus (e.g. by forming at least part of the articulated robot arm of suitable heat sink materials), which may help provide a relatively compact and convenient arrangement. Such an arrangement may also allow for improved range of movement, e.g. compared to mounting a separate block of heat sink material to the articulated robotic arm. Accordingly, the heat sink is not limited to any particular shape or size, provided that it may still function to draw heat from the generator unit.

The heat sink is positioned for thermal communication with the electrosurgical generator unit when in use, so that it may conduct heat away from the electrosurgical generator unit. Thermal communication may be achieved either directly (i.e. by providing direct physical contact between the heat sink and the electrosurgical generator unit), or indirectly (i.e. permitting a physical gap between the heat sink and electrosurgical generator unit). In embodiments utilising indirect thermal communication, it may be preferable for the cooling arrangement to include a thermally conductive element (e.g. heat pipe or thermally conductive material) to at least partially bridge the gap (e.g. by contacting a connector such as a socket for holding the electrosurgical generator unit) to thereby improve the thermal coupling between the heat sink and electrosurgical generator unit when in use.

As an example, direct thermal communication may be achieved by mounting the heat sink directly onto a surface of the electrosurgical generator unit, or a portion of the robotic surgical tool which physically contacts the generator unit when mounted. Additionally or alternatively, the heat sink may be provided on a portion of the connector which physically contacts the electrosurgical generator unit when mounted.

Indirect thermal communication may be achieved by positioning a heat sink in thermal communication with the connector. The connector may be formed of a thermally conductive material (or may include a thermal link, e.g. heat pipe) to thermally couple the heat sink and the electrosurgical generator unit when in use.

The cooling assembly may include a thermally conductive element to thermally couple the heat sink to the electrosurgical generator unit. The thermally conductive element may comprise a material having a high thermal conductivity, e.g. a metal element such as aluminium, copper, brass. Alternatively or additionally, the cooling assembly may comprise a heat pipe for thermally coupling between the heat sink and the electrosurgical generator unit when the electrosurgical generator unit is mounted to the robotic surgical tool.

A heat pipe is an efficient thermal conductor and may be considered a type of thermally conductive element, which may help to draw heat away from the electrosurgical generator unit and toward the heat sink. The heat pipe may be arranged to provide a direct connection between the heat sink and the electrosurgical generator unit (by directly contacting both units), or an indirect connection (i.e. by forming only a portion of a thermal pathway between the electrosurgical generator unit and the heat sink). These direct and indirect couplings are similar to those discussed above, e.g. with reference to the heat sink. An indirect connection may be achieved, for example, by the heat pipe contacting the heat sink but not physically contacting the electrosurgical generator (and instead contacting e.g. a separate connector).

In summary, one or more heat sinks and/or thermally conductive elements may be provided, having materials properties (e.g. heat capacity and/or thermal conductivity) selected to help promote heat dissipation from the electrosurgical generator unit. These may be considered forms of passive heat dissipation.

Alternatively or in combination, passive heat dissipation may also be provided by configuring a shape of the robotic surgical tool and/or electrosurgical generator unit to improve heat transfer.

For example, the robotic surgical tool may include the connector, which may be in the form of a recess (or cavity or socket) formed in the robotic surgical tool, where the recess is dimensioned to provide a close fit to the electrosurgical generator unit. For example, the recess may cover a majority or entirety of an outer surface of the electrosurgical generator unit, to promote heat transfer from the electrosurgical generator unit to the robotic surgical tool. The recess may be sized and shaped to provide a transition fit or interference fit to the housing of the robotic surgical tool. This may provide close thermal contact between the electrosurgical generator unit and the robotic surgical tool, thereby improving heat flow therebetween. In an embodiment, the connector may have a static portion (e.g. a recess) and a moveable portion (e.g. a door). In use, the moveable portion may be moved into an open position so that the generator unit can be inserted into the static portion and then, once inserted, the moveable portion may be moved to a closed position so that the generator unit is held between the static and moveable portions.

Optionally, the cooling assembly may comprise a resilient, thermally conductive connector for positioning between the robotic surgical tool and the electrosurgical generator unit to press against the robotic surgical tool and the electrosurgical generator unit when the electrosurgical generator unit is mounted to the robotic surgical tool. The thermally conductive connector may help improve the thermal coupling between the robotic surgical tool and the electrosurgical generator unit. The resilient nature of the connector may help to provide a tight fit to improve thermal contact between the electrosurgical generator unit and robotic surgical tool, while the thermal conductivity helps to dissipate heat. For example, the resilient connector may include one or more springs formed of thermally conductive material (e.g. metal).

In an embodiment, the connector for mounting the electrosurgical generator unit to the robotic surgical tool comprises the resilient, thermally conductive connector.

Optionally, the cooling assembly may comprise one or more fins arranged to be in thermal communication with the electrosurgical generator unit when the electrosurgical generator unit is mounted to the robotic surgical tool. Fins help to increase the surface area of the apparatus, thereby facilitating heat dissipation. The fins may be provided on an external surface of the electrosurgical generator unit, the connector and/or the robotic surgical tool which is in thermal communication with the electrosurgical generator unit (e.g. on a surface of the articulated robot arm close to the connector). Additionally or alternatively, fins may be provided on an external heat sink configured to thermally couple to the electrosurgical generator unit. In this way, the fins may promote heat dissipation from the heat sink to indirectly increase the rate of heat transfer from the generator unit to the heat sink.

The above-discussed cooling arrangements may be considered ‘passive’ cooling arrangements, meaning that they do not require a power source (e.g. electrical power source) in order help dissipate heat. Additionally or alternatively, the cooling assembly may comprise an electrically controlled active cooling mechanism for actively cooling the apparatus.

The active cooling mechanism may be configured to cool an element of the apparatus that is in thermal communication with the electrosurgical generator unit (e.g. the robotic surgical tool). This indirect cooling may serve to increase a temperature gradient between the cooled element (e.g. robotic surgical tool) and the (hotter) electrosurgical generator unit, thereby promoting a preferential heat flow from the electrosurgical generator unit toward the cooled element (e.g. robotic surgical tool). Alternatively, the active cooling mechanism may be configured to directly cool the electrosurgical generator unit itself, e.g. by incorporating a fan within the electrosurgical generator unit or by configuring a fan to direct airflow over an outer surface (e.g. housing) of the generator unit.

Preferably, the robotic surgical tool comprises an energy delivery structure to provide electrical power to the active cooling mechanism. In other words, the energy delivery structure (e.g. a cable or inductive link) which provides power to the active cooling mechanism may be incorporated into the robotic surgical tool itself (e.g. in the control column and/or articulated arm). This arrangement may help avoid the need for separate cables to supply power to the active cooling mechanism, thereby helping to save space in the operating theatre and reduce the risk of trip hazards over separate leads.

Preferably, arrangements having an active cooling mechanism may further comprise a sensor for detecting a temperature of the apparatus; and a controller to control the active cooling mechanism based on the detected temperature. For example, the control unit may be configured to vary a power level of the active cooling mechanism based on a sensed temperature at the element which the cooling mechanism is configured to cool. Thus, the active cooling is provided in response to substantially real-time feedback indicating the status of the apparatus. In an embodiment, the control unit may be configured to vary a power level of the active cooling mechanism by activating/de-activating the active cooling mechanism, i.e. by switching the active cooling mechanism between “on” and “off” power levels.

In another embodiment, the control unit may be configured to vary the power level in different increments (e.g. low, medium, high power levels) at the active cooling mechanism.

For example, the sensor may be configured to detect a temperature of the electrosurgical generator unit, and the control unit may control an active cooling mechanism (e.g. a fan) based on the detected temperature. This may help to avoid overheating at the electrosurgical generator unit when it is in use, and avoid unnecessarily cooling the electrosurgical generator unit when it is not being used (for example, because the surgeon is taking a break from using the electrosurgical instrument).

Alternatively, the sensor be configured to detect a temperature of another element, e.g. a heat pipe thermally connectable to the electrosurgical generator unit. This may help to ensure that the end of the heat pipe furthest from the electrosurgical generator unit is at a sufficiently low temperature to cause the fluid in the pipe to transition from vapour to liquid, so that the heat pipe may function as required (further details of which are described below).

In some other embodiments, the sensor may be configured to detect a temperature of the connector, the robotic surgical tool (e.g. a portion that is close to the generator unit when mounted) and/or the cooling assembly (e.g. a portion that is close to the generator unit when mounted).

In embodiments, the controller may be configured to control a temperature level of the active cooling mechanism in response to a detected temperature, e.g. by controlling a temperature of a coolant fluid supply.

Preferably, the control unit includes a watchdog unit to issue a notification if a temperature exceeds an acceptable threshold. The notification may be issued to the control unit, which may respond by varying a power level of the active cooling mechanism, activating an additional active cooling mechanism, and/or powering off the apparatus to help ensure safety of the system.

Optionally, the active cooling mechanism may comprise a fan to direct air flow over a surface of the apparatus. For example, the fan may direct air flow over a surface of the robotic surgical tool, electrosurgical generator unit, the connector or another element of the cooling arrangement (e.g. a separate heat sink).

Alternatively or additionally, the active cooling mechanism may comprise a heat pump to remove heat from the apparatus. For example, the heat pump may comprise a thermoelectric heat pump, e.g. a Peltier cooler. The heat pump may help to remove heat from the apparatus, for example, the robotic surgical tool, the electrosurgical generator unit, the connector, or another element of the cooling arrangement (e.g. a separate heat sink). For example, the heat pump may be connected to a heat sink of the apparatus to remove heat from the heat sink.

Optionally, the cooling arrangement may use a fluid to draw heat from the apparatus. This may be achieved passively, e.g. by using a heat pipe, or by submerging an element of the apparatus into a bath of coolant fluid (e.g. submerging a separate block of heat sink material thermally connectable to the electrosurgical generator unit). Alternatively, an active cooling mechanism may be employed to cool the apparatus by using a fluid. For example, the apparatus may comprise one or more conduits arranged to be in thermal communication with the electrosurgical generator unit when the electrosurgical generator unit is mounted to the robotic surgical tool, wherein the active cooling mechanism is configured to circulate a coolant fluid through the one or more conduits. For example, the conduits may be in direct or indirect thermal communication with the connector for mounting the electrosurgical generator unit to the robotic surgical tool. In this manner, heat may be effectively removed by the coolant fluid as it circulates through the one or more conduits.

The conduits may be configured for thermal communication with the electrosurgical generator unit either directly (e.g. by being formed as channels in the electrosurgical generator unit itself) or indirectly (e.g. by being formed as channels in the robotic surgical tool, or as conduits/pipes around the robotic surgical tool or electrosurgical generator unit). The one or more conduits may define an irregular or undulating path. This may help to increase the area of fluid that can be in thermal communication with the electrosurgical generator unit, so that heat can be more effectively removed.

The active cooling mechanism may comprise a pump or other suitable mechanism for causing the coolant fluid to flow through the one or more conduits, for example, from a cooling fluid reservoir. Optionally, a control unit may control the pump (or other mechanism) to vary a flow rate of the fluid through the one or more conduits based on a detected temperature. This control unit may be the aforementioned controller which controls active cooling based on sensor temperature.

Preferably, the robotic surgical tool comprises the one or more conduits. By incorporating the conduits into the robotic surgical tool, this may provide similar advantages as discussed above and in the priority application, i.e. by helping to save space in the operating theatre and reduce the risk of trips over separate conduits. Optionally, the robotic surgical tool may comprise a thermally insulating outer layer having one or more holes to accommodate the one or more conduits for circulating coolant fluid within the robotic surgical tool. The conduits may therefore help to draw heat from the electrosurgical generator unit, through the robotic surgical tool, whilst the thermally insulating layer may help to guard a user from the excess heat.

In some embodiments, the robotic surgical tool comprises the cooling assembly, e.g. by incorporating/integrating the cooling assembly into the articulated robot arm and/or a control column of the robotic surgical tool. This provides a relatively convenient and compact arrangement, since the cooling assembly forms part of the robotic surgical tool itself, without requiring additional separate components.

In variant embodiments, the cooling assembly may be provided as a separate component which is thermally connectable to the robotic surgical tool and/or electrosurgical generator unit. By providing a cooling arrangement which is not integrated in the robotic surgical tool, the cooling arrangement can be positioned to dissipate heat even further away from a patient. Further, such an arrangement may also be interchangeably connectable for retrofitting to a plurality of surgical apparatus.

Alternatively or in combination, a cooling arrangement may be incorporated in the electrosurgical generator unit itself. Accordingly, a second aspect of the invention provides an electrosurgical generator unit for mounting to a robotic surgical tool, the electrosurgical generator unit comprising: a housing; a signal generator contained within the housing, the signal generator being configured to generate an electrosurgical signal for use by an electrosurgical instrument supported by the robotic surgical tool; an energy delivery structure to couple the electrosurgical signal into the robotic surgical tool; and a cooling assembly for dissipating heat produced by the electrosurgical generator unit.

By including the cooling assembly as part of the electrosurgical generator unit, wherein the electrosurgical generator unit is detachably mountable on the robotic surgical tool and has an energy delivery structure for coupling to the robot-assisted surgical system, this provides a convenient and integrated generator unit which can be used in a variety of robotic surgical systems whilst helping to maintain appropriate temperatures and reduce the risk of overheating.

The optional features discussed above in relation to the first aspect of the invention are equally applicable to the second aspect of the invention, i.e. they can also be provided as part of the electrosurgical generator unit alone.

For example, the electrosurgical generator unit may comprise a heat sink to conduct heat away from one or more elements of the electrosurgical generator unit. For example, the heat sink may be mounted on the housing of the electrosurgical generator unit to draw heat away from an internal component of the electrosurgical generator unit (e.g. the signal generator, a processor, control unit, or other module).

Optionally, the cooling arrangement may include a heat pipe to thermally couple between the heat sink and the one or more elements (e.g. the housing, the signal generator, a processor, control unit, or other module).

The cooling arrangement may include a resilient, thermally conductive connector on the housing of the electrosurgical generator unit, for pressing against the robotic surgical tool when the electrosurgical generator unit is mounted to the robotic surgical tool.

The housing may include one or more fins for dissipating heat from the electrosurgical generator unit.

The electrosurgical generator unit may comprise an electrically controlled active cooling mechanism for actively cooling a portion of the electrosurgical generator unit. The active cooling mechanism may be configured to receive power from an internal battery of the electrosurgical generator unit. Alternatively, the active cooling mechanism may be coupled to an input for receiving power from an energy delivery structure of the robotic surgical tool.

The active cooling mechanism may include a fan to direct air flow over a surface of the electrosurgical generator unit, e.g. inside the electrosurgical generator unit.

The active cooling mechanism may include a heat pump, e.g. mounted to the housing, to remove heat from the electrosurgical generator unit.

The electrosurgical generator unit may comprise one or more conduits arranged to be in thermal communication with the one or more elements of the electrosurgical generator unit (e.g. the housing, the signal generator, the processor, control unit, or other module), and the active cooling mechanism may be configured to circulate a coolant fluid through the one or more conduits. For example, the one or more conduits may be formed as channels in the housing of the electrosurgical generator unit or as conduits/pipes around the electrosurgical generator unit.

The invention includes the combination of the aspects and preferred features described except where such a combination is clearly impermissible or expressly avoided.

### SUMMARY OF THE FIGURES

Embodiments of the invention are described in detail below with reference to the accompanying drawings, in which:

FIG. 1 is an overall schematic system diagram of a robot-assisted electrosurgical system to which the present invention is applied;

FIG. 2 is a perspective view of an articulated robotic arm for electrosurgery;

FIG. 3 is a schematic diagram of an instrument holder for an articulated robotic arm;

FIG. 4 is a schematic diagram of a removable electrosurgery capsule for a robotic surgical tool;

FIG. 5 is a schematic diagram of a removable electrosurgery capsule for a robotic surgical tool, the electrosurgery capsule having a cooling arrangement according to an embodiment of the invention;

FIG. 6 is a schematic diagram of a robotic surgical tool and a cooling arrangement according to an embodiment of the invention;

FIG. 7 is a schematic diagram of a robotic surgical tool and a cooling arrangement according to an embodiment of the invention; and

FIG. 8 is a schematic diagram of an electrosurgical instrument that can be handled by an articulated robotic arm in an embodiment of the invention.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The present invention relates to the context of robot-assisted surgery.

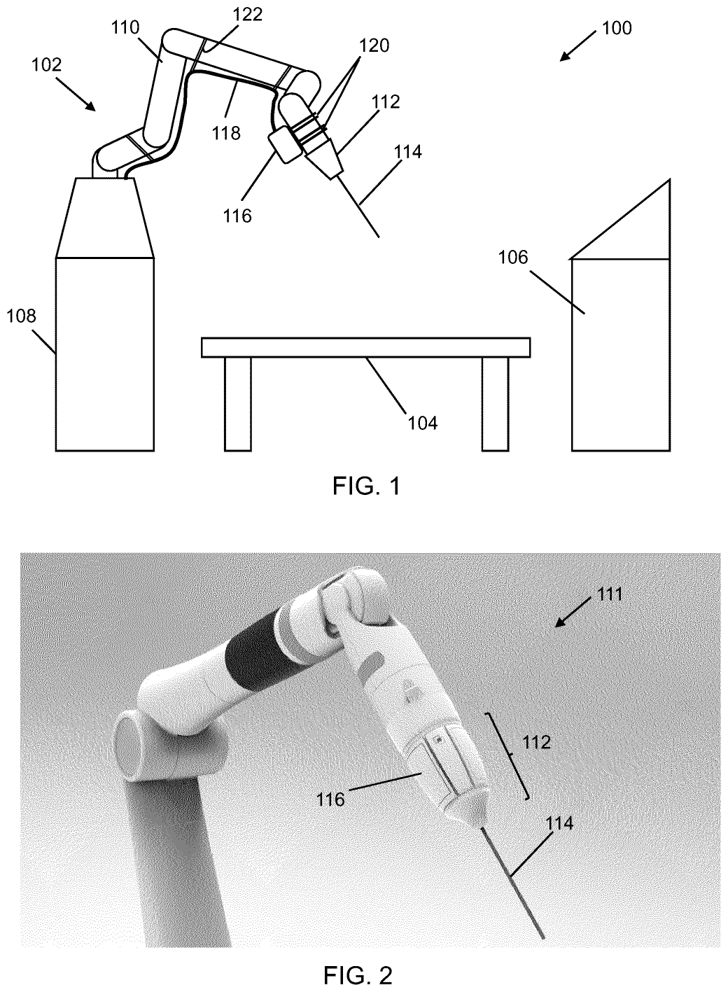

As discussed above, the disclosure herein relates to developments of a concept put forward in the applicant's earlier GB patent application no. 2012303.0, filed on 7 Aug. 2020, and incorporated herein by reference. Examples of the robot-assisted electrosurgical system may be understood with reference to FIGS. 1 to 4, described below.

FIG. 1 is an overall schematic system diagram of a robot-assisted electrosurgical system 100 to which the present invention may be applied. The system 100 comprises three main entities: a robotic surgical tool 102, an operating table 104, and a control console 106. The operating table 104 provides a location for receiving a patient for a procedure with which the robotic surgical tool 102 can assist.

In this example, the robotic surgical tool 102 comprises a control column 108 having an articulated arm 110 extending therefrom. The control column 108 may support a plurality of articulated arms. An instrument holder 112 is mounted at a distal end of the articulated arm 110. The articulated arm 110 may be considered to comprise the instrument holder 112. The instrument holder 112 is configured to hold a surgical tool 114.

In this example, the surgical tool 114 is depicted as a rigid elongate element, suitable for insertion into the patient's body e.g. using known laparoscopic techniques or the like. The articulated arm 110 allows the position and angle of the surgical tool 114 relative to the operating table 104 to be varied. The control column 108 may also be movable within the operating theatre environment.

The instrument holder 112 may comprise various ports that are connectable to the surgical instrument 114. For example, the instrument holder 112 may provide a link through which an end effector of the surgical instrument 114 can be controlled. The instrument holder 112 may also be used to deliver power or other substances (e.g. saline or the like) to the surgical instrument.

The control console 106, which is typically in the same room as the operating table 104 and robotic surgical tool 102, is normally separate from the robotic surgical tool 102 and is used to remotely control the articulated arm 110 and instrument holder 112. The articulated arm 110 may also be positioned manually.

The robotic surgical tool 102 is provided with a detachable electrosurgery capsule 116 that is configured to generate and deliver, via the instrument holder 112, electrosurgical signals for use by the surgical instrument 114. In this example, the electrosurgery capsule 116 is secured to the articulated arm 110 by one or more suitable connections 120, e.g. straps or the like. However, in other examples discussed herein, the electrosurgery capsule 116 may be directly connectable to the instrument holder 112, e.g. as a plug-in module. In yet other examples, the electrosurgery capsule 116 may be secured to the control column 108 of the robotic surgical tool 102, rather than being secured to the articulated arm 110.

The electrosurgery capsule 116 may be a stand-alone unit for generation and delivery of signals suitable for use in electrosurgery. As discussed in more detail in the applicant's earlier GB patent application no. 2012303.0, the electrosurgery capsule 116 may be powered by an internal DC supply of the robotic surgical tool 102. That is, the robotic surgical tool 102 may be connected to a mains electricity supply in a standard manner (not shown). The control column 108 may include circuitry to transform the mains supply into a DC supply for use by the robot. The control column 108 may have a first DC supply for controlling movement of the articulated arm 110. Typically, the first DC supply may have a voltage of 24 V and permit current up to 2 A. The control column 108 may provide a second DC supply for use by or at the instrument holder 112. The second DC supply may have the same voltage (e.g. 24 V) or a lower voltage (e.g. 12 V) than the first DC supply, The second DC supply may have a more limited current supply (e.g. no more than 600 mA). The electrosurgery capsule 116 may utilise either the first or second DC supply.

In the example shown in FIG. 1, the electrosurgery capsule 116 is connected to the control column 108 by a separate cable 118, which may be retained on the articulated arm 110 by one or more clips 122. The cable 118 may convey a DC signal from the first DC supply. Alternatively or additionally, the electrosurgery capsule 116 may be arranged to receive power via the same route as the instrument holder 112.

FIG. 2 is a perspective view of an articulated robotic arm 111 for electrosurgery. Features in common with the system of FIG. 1 are given the same reference number. In this example, the articulated robotic arm 111 performs the same function as the articulated robotic arm 110 of FIG. 1. However, instead of having one or more electrosurgery capsules 116 attached to an outer surface thereof, the articulated robotic arm 111 has an instrument holder 112 provided with a recess or socket configured to receive an electrosurgery capsule 116. The electrosurgery capsule 116 may be detachably mountable in the recess, e.g. to permit it to be readily exchanged for electrosurgery capsule that provides a different modality, or to permit the electrosurgery capsule 116 to be switched to another articulated robotic arm 111 on the same or a different control column.

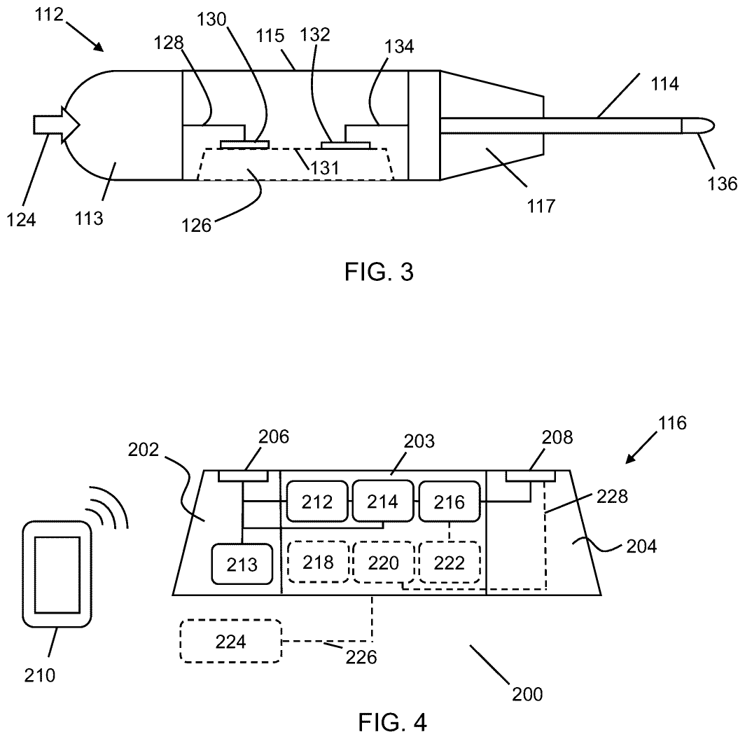

FIG. 3 is a schematic diagram of an instrument holder 112 for an articulated robotic arm 111 of the type shown in FIG. 2. The instrument holder 112 may have any suitable shape, although in the example it has a generally cylindrical form, extending along a longitudinal axis that is aligned with the surgical instrument 114 that extends from a distal portion 117 thereof.

The instrument holder 112 comprises a proximal portion 113 that is attached to (and may pivot on) a distal end of the articulated robotic arm. The proximal end 113 may be configured to receive a power input 124 that is conveyed through the articulated robot arm.

In this example, the instrument holder 112 comprises an intermediate portion 115 that has a recess 126 formed therein. There may be a plurality of recesses formed around a circumference of the intermediate portion. The instrument holder 112 may thus be configured to receive one or more electrosurgical capsules within the recesses 126.

The intermediate portion 115 may further comprise means for interconnecting the electrosurgery capsule. For example, the recess 126 may have one or more input/output ports mounted on an internal surface thereof. In the example shown in FIG. 3, there is an input port 130 configured to deliver power (e.g. a DC signal) into the electrosurgery capsule. The input port 130 is connected to the proximal portion 113 by a suitable transmission line 128 that in turn is connected to the power input 124. Similarly, there is an output port 132 configured to deliver the electromagnetic signal (e.g. radiofrequency or microwave energy) from the electrosurgery capsule to the surgical instrument 114. The output port 132 may be connected to the distal portion 117 by a suitable transmission line 134 (e.g. a coaxial cable). The distal portion 117 may be configured with a suitable connector (e.g. QMA connector or the like) to connect the transmission line 134 to an energy conveying structure (e.g. another coaxial transmission line) within the surgical instrument 114 itself. An example of this is discussed below with reference to FIG. 8.

The surgical instrument 114 may be detachably mounted to the distal portion 117. The same instrument holder 112 may thus be used with a plurality of instruments. Moreover, in the invention the instrument holder 112 may be used with a plurality of different types of electrosurgery capsule. This enables a variety of combinations of instrument and energy modality to be used interchangeably at the same instrument holder.

FIG. 4 is a schematic diagram of a removable electrosurgery capsule 116 (also referred to as an “electrosurgical generator unit”) for a robot arm. The electrosurgery capsule 116 may be receivable in a recess 126 of the type discussed above.

The electrosurgery capsule 116 comprises a rigid housing 200, which may be shaped to cooperate with a recess in the instrument holder of a robotic arm a manner that aligns the capsule appropriately. The electrosurgery capsule 116 includes an input portion 202 that is communicably connectable to a control network of the robot-assisted surgical system, e.g. via the instrument holder. The input portion 202 may also be configured to receive a power supply (e.g. an internal DC supply of the instrument holder). The electrosurgery capsule 116 further includes an operational portion 203 that houses various functional components or modules for generating and/or controlling an electromagnetic signal, and an output portion 204 for delivering the electromagnetic signal into the instrument holder, from where it is conveyed to an electrosurgical instrument held by the robotic arm.

In this example, the input portion 202 comprises an input connector 206 for receiving input control and power signals. The output portion 204 may comprise an output connector 208 for delivering a generated electromagnetic signal out of the electrosurgery capsule 116. In general, the electrosurgical capsule referred to herein is configured to produce electromagnetic (EM) radiation, such as radio frequency (RF) or microwave EM radiation, suitable for treating or measuring biological tissue.

Various electrosurgical modalities are presented below in the context of a robot-assisted surgical system for use in laparoscopic or endoscopic procedures involving the controlled delivery of EM energy, for example, RF and microwave energy. Such EM energy may be useful in the removal of polyps and malignant growths. However, it is to be understood that the aspects of the invention presented herein need not be limited to this particular application. Also, they may be equally applicable in embodiments where only RF energy is required, or where only RF energy and fluid delivery is required.

Returning to FIG. 4, the operational portion 203 of the electrosurgical capsule 116 in this example is configured as a modular system that includes a plurality of modules. The plurality of modules includes a controller module 212, a signal generator module 214, and a feed structure module 216. These may be the core modules of the operational portion 203. These core modules provide mechanisms for controllably generating an EM signal for treating biological tissue, and for delivering that EM signal to an electrosurgical instrument. The EM signal may be any type of electromagnetic signal, such as RF or microwave.

Additionally, the plurality of modules may include further optional modules such as a signal detector module 218, a fluid feed module 220, and/or one or more additional signal generator modules 222. The optional nature of these modules is indicated in FIG. 4 by dashed lines. In some examples, different optional modules may be combined together with core modules to provide a capsule with different electrosurgical capabilities.

Further details of the electrosurgical capsule and its modules are provided in the applicant's earlier GB patent application no. 2012303.0. Briefly summarised, control of the capsule (or of a plurality of capsules) is done using control signals delivered via a control network of the robot-assisted surgical system (e.g. using the control console or a remote computing device 210) to the controller module 212 within the capsule 116. The controller module 212 communicates with each other module of the electrosurgery capsule 116 and can control each other module via control commands. The signal generator module 214 is operable to generate and control EM radiation based on the control commands to form an EM signal for treatment of biological tissue. The signal generator module 214 may be powered by a signal received from the input connector 206 and/or by an internal battery 213. The feed structure module 216 provides an energy delivery structure configured to couple the EM signal into the surgical instrument via the robotic arm.

The electrosurgery capsule need not require a dedicated connection to an external main supply. This may be desirable, as it obviates the need to consider means for isolating the mains supply from the surgical instrument and, ultimately, the patient.

The fluid feed module 220 is controllable by the controller module 212 based on the control commands to supply and control a flow of fluid (e.g. gas or liquid) via the fluid feed structure 228 to the output connector 208. The purpose of the fluid feed module 220 may be to provide fluid to the distal instrument tip 136. For example, the fluid may be a gas which is provided to the surgical instrument 114 for generating plasma for treatment of biological tissue, or the fluid feed module 220 may provide liquid (e.g. saline) to the distal instrument tip 136.

The fluid feed module 220 includes a fluid feed structure 228 in fluid communication with a fluid port for outputting fluid to the surgical instrument 114. In this example, the fluid feed structure 228 delivers fluid to the output connector 208, where it may be conveyed to the surgical instrument via a suitable coupling within the instrument holder 112. The energy delivery structure and the fluid feed may be combined in a common feed structure. For example, the transmission line 134 within the instrument holder 112 may be configured as a combined fluid and energy feed to deliver both the fluid and the EM energy to the surgical instrument 114. The surgical instrument 114 may in turn comprise a fluid feed that transport fluid to the distal instrument tip 136.

A fluid supply 224 (e.g. a pressurised gas canister or the like) may be mounted on an external surface of the electrosurgery capsule 116. The fluid feed module 220 may connected to the fluid supply 224 by a feed conduit 226.

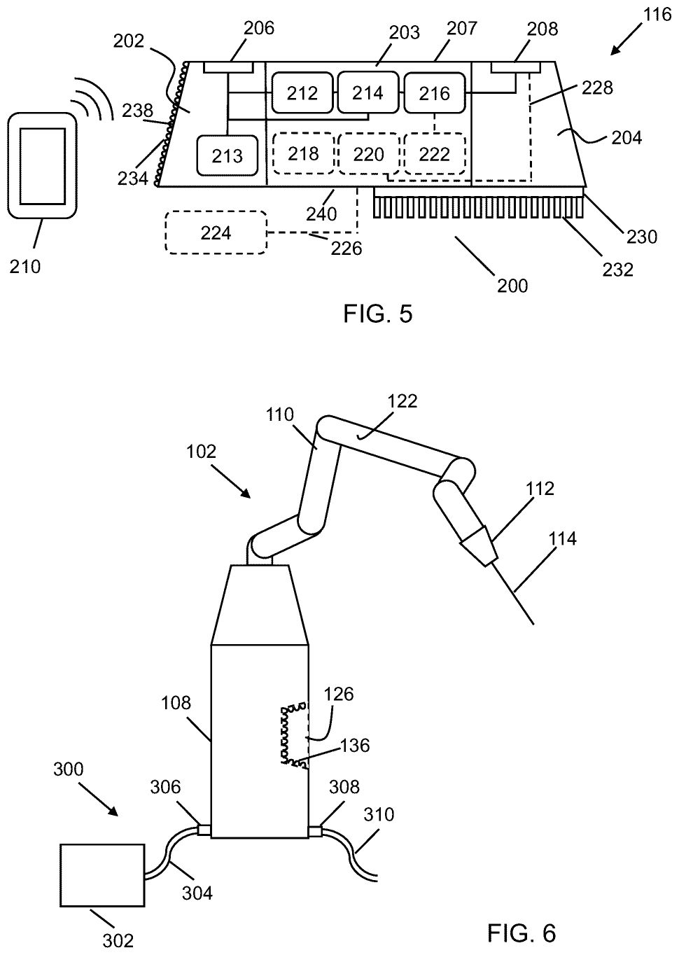

FIG. 5 shows a removable electrosurgery capsule (electrosurgical generator unit) 116 having a cooling arrangement for dissipating heat produced by the capsule 116, according to an embodiment of the invention. The cooling arrangement includes passive and active cooling mechanisms, including a heat sink 230, fins 232, thermally conductive connectors 234, and a fan (not illustrated). Each of these cooling mechanisms help to dissipate heat from the electrosurgical generator unit. Other embodiments may include different cooling mechanisms alone or in various combinations.

Similarly to the capsule 116 of FIG. 4, the capsule 116 of FIG. 5 is configured to be received within the recess 126 of the instrument holder 112 (see FIG. 3). Specifically, the housing 200 of the capsule 116 is configured to be mounted within the recess 126, e.g. via snap-fit engagement.

Although described here in the context of a recess within an instrument holder, in variant embodiments, the recess may be formed in another portion of the robotic surgical tool, e.g. within the control column, as described further herein.

In FIG. 5, the input connector 206 and output connector 208 are located at a rear surface 207 of the capsule 116. When mounted within the recess 126, the input and output connectors 206, 208 are brought into connection with respective input and output ports 130, 132, which are at a complementary rear surface 131 of the recess 126.

The housing 200 further includes a peripheral outer surface for locating within a complimentary peripheral inner surface of the recess 126. In this embodiment, the peripheral outer surface forms four side walls 238 (see FIGS. 2 and 5). In the embodiment of FIG. 5, resilient, thermally conductive connectors 234 (e.g. metal springs) are provided on the outer surface of one of the side walls 238. These connectors 234 are configured to engage against a complementary inner wall of the recess 126 when in use. In doing so, the connectors 234 help to provide a close fit within the recess 126, effectively pushing an opposing side wall 238 of the capsule closer against the recess 126. Therefore, the connectors 234 increase the thermal coupling between the housing 200 and the recess 126 in the instrument holder 112, to facilitate heat flow from the capsule 116 toward the instrument holder 112. This may help to reduce the risk of overheating of the capsule 116 (e.g. at its processor or signal generator). Therefore, malfunction of the capsule may be avoided.

In addition to the rear surface 207 and side walls 208, the housing 200 further includes a front surface 240 which faces outwardly away from the slot 126 when in use. In FIG. 5, the front surface 240 is provided with a cooling arrangement, which may dissipate heat away from both the capsule 116 and the instrument holder 112. The cooling arrangement at the front surface 240 includes a heat sink 230 with fins 232 formed thereon. The heat sink 230 is a block of material having a high thermal conductivity (e.g. metal), to conduct heat away the remaining components of the capsule 116. The fins 232 are also formed of a thermally conductive material and define channels for permitting airflow through the fins to help dissipate heat.

In combination, the instrument holder 112 may also include a cooling arrangement (e.g. a heat sink), which may be positioned to draw heat in a direction away from the surgical instrument 114.

In this embodiment, the housing 200 is itself formed of a thermally conductive material (e.g. metal) to help conduct heat from the capsule 116. The housing 200 may therefore be considered an additional heat sink. In variant embodiments, which are not illustrated, fins may be formed directly onto a surface of the housing 200 (omitting the additional heat sink 230). In yet further variant embodiments, the housing may be formed of a thermally insulating material and may include a thermally conductive element extending therethrough to thermally couple an external heat sink (e.g. heat sink 230) to the internal components of the capsule.

In summary, the connectors 234, heat sink 230, and fins 232 are each passive cooling arrangements to promote directional heat flow without requiring any power to be applied. This helps to mitigate overheating stemming e.g. from processing at the controller module 212 or from the signal generator module(s) 214, 222. In use, when internal components of the capsule 116 begin to overheat, excess heat will flow towards regions of lower temperature, where the passive cooling means are provided to help absorb or dissipate that heat.

The capsule 116 further includes an active cooling mechanism in the form of an internal fan (not shown) to cool the internal components of the capsule 116, e.g. by cooling a processor or memory of the controller module 212. The internal fan may receive power from the surgical tool via the input connector 206, and/or may receive power from the battery 213. This arrangement helps to avoid malfunction of internal components caused by overheating, without requiring separate external cables to power the fan.

Therefore, in this embodiment, the active cooling mechanism (fan) is internal to the capsule. In variant embodiments, an active cooling mechanism may be configured to cool an element or surface that is external to the capsule 116. Such external cooling may be useful to increase a thermal gradient between the inside and outside of the capsule, thereby helping to increase directional heat flow away from the inside of the capsule.

In FIG. 5, the controller module 212 includes a watchdog (or fault detection unit) for monitoring a range of potential error conditions which could result in the system not performing to its intended specification. Further, the controller module 212 includes one or more sensors which monitor the operation of various parts of the system. The watchdog may generate alarm signals when the outputs of these sensors moves outside of preset limits. For example, the controller module 212 may include one or more temperature sensors operable to generate temperature measurements based on a temperature of part of the controller module 212, such as, the processor or memory of the controller module 212. The watchdog may then be operable to generate an alarm signal based on a comparison between the temperature measurements and one or more preset temperature limits, to indicate that the part is overheating. The controller module 212 may control a power level of the active cooling mechanism based on the detected temperature. For example, the controller module 212 may control the power level of the fan in order to maintain a detected temperature within a target range. For example, the watchdog may issue a notification indicating that the temperature has exceeded a threshold value, and the controller module 212 may respond by increasing a power level of the active cooling mechanism, or by activating one or more additional active cooling mechanisms.

Additionally or alternatively, a different type of sensor (e.g. a voltage or current sensor) may be provided to monitor the operation of the active cooling mechanism, such that the watchdog generates an alarm signal if the sensor indicates that the active cooling mechanism (e.g. fan) has malfunctioned (e.g. it is using no voltage or current). Additionally or alternatively, a sensor may monitor a voltage level of a DC power supply of the controller module 212, and the watchdog may generate an alarm signal if the voltage level drifts out of a predetermined accepted range of operation.

It is to be understood that the controller module 212 can contain different types of sensors which monitor the operation of different elements of the controller module, and the watchdog may monitor the outputs of these sensors and generate an alarm signal if any one of these outputs moves outside of preset limits. Additionally, the controller module 212 may contain sensors which monitor the operation of other modules, and the watchdog may monitor the outputs of these sensors and generate an alarm signal if any one of these outputs moves outside of preset limits. Additionally, the sensors and watchdog unit may be provided as part of another element of the robotic surgical system, e.g. as part of the robotic surgical tool rather than the generator. In this case, a separate controller may be provided to receive the sensed temperature and/or watchdog alerts, and to control the active cooling mechanism in response. Such an arrangement may be particularly convenient if the active cooling mechanism is provided as part of the robotic surgical tool rather than as part of the generator unit. By providing the cooling mechanism, sensors, and watchdog externally to the capsule, the power requirements at the capsule may be reduced, thereby further reducing the risk of overheating at the capsule.

The controller module 212 may handle an alarm signal in a number of different ways. For example, the controller module 212 may cause the watchdog to transmit the alarm signal via the wireless communication interface to the remote computing device 210. In this way, the remote computing device 210 can keep a record or log of when faults occur. Also, the watchdog may include in the alarm signal a reference to a type of fault to which the alarm signal relates such that the remote computing device 210 can include this information in the log. Also, the remote computing device 210 may externally control the response of the capsule based on the alarm signal. For example, the remote computing device 210 may send particular control commands to the controller module 212 based on the alarm signal, for example, so as to shut down the electrosurgical capsule 116 in a safe manner. In this way, the remoted computing device 210 may externally control the response of the electrosurgical capsule 116 based on the alarm signal. Additionally or alternatively, the controller module 212 may itself generate control commands based on the alarm signal. In this way, the controller module 212 may internally control the response of the electrosurgical capsule 116 based on the alarm signal.

In an embodiment, where the controller module 212 includes the processor, the watchdog includes an independent processor (e.g. a microprocessor) so that the watchdog can confirm that the processor is functioning correctly, i.e. raise an alarm signal if the processor malfunctions (e.g. uses no voltage or current). Alternatively, the watchdog may be implemented in software which is executed by the processor of the controller module 212, i.e. no separate hardware processor may be included.

FIG. 6 is a schematic diagram of a robotic surgical tool 102 according to an embodiment of the invention.

The robotic surgical tool 102 of FIG. 6 is similar to that previously described but differs in two main respects. Firstly, the control column 108 of FIG. 6 includes a recess 126 for receiving the capsule, rather than strapping the capsule to the articulated arm (as shown in FIG. 1) or inserting the capsule into a recess of the instrument holder (as shown in FIG. 2). Secondly, the robotic surgical tool 102 of FIG. 6 is provided with a cooling arrangement for dissipating heat from the capsule.

The recess 126 of the control column 108 is largely similar to that previously described with reference to the instrument holder 111. However, in FIG. 6, the recess 126 is provided with a cooling arrangement in the form of resilient, thermally conductive connectors 136 (e.g. metal springs) at its inner surface. The connectors 136 function in a similar manner to the connectors 234 previously described with reference to FIG. 5, by providing a close fit with the capsule when it is inserted in the recess 126. The walls of the recess 126 are formed of a thermally conductive material to allow heat transfer into the control column 108.

The cooling arrangement in FIG. 6 further includes a fluid system 300 for circulating a coolant fluid through a conduit in the control column 108 which is in thermal communication with the walls of the recess 126, to draw heat from the electrosurgical generator unit 116 when in use. Specifically, the fluid system 300 includes a coolant fluid source 302 for supplying a coolant fluid. The fluid system 300 further includes an inlet conduit 304 fluidically connected to the coolant fluid source 302, to carry coolant fluid from the coolant fluid source 302 through an inlet 306 formed in the control column 108. Inside the control column 108, a coolant conduit (not shown) is fluidically connected from the inlet 306 to an outlet 308, to provide a fluid-tight flow path for circulating the coolant fluid through the control column 108. An outlet conduit 310 is fluidically connected to the outlet 308 to carry the coolant fluid (and consequently any heat that it has absorbed) away from the apparatus. This heated coolant fluid may then be cooled (e.g. via active and/or passive cooling means) and reintroduced to the source 302 for reuse.

The coolant system 300 (e.g. the fluid source 302) may include a pump or other mechanism to control a flow rate of fluid through the coolant system. The pump may be controlled by a controller to vary the flow rate of the coolant fluid in response to a measured temperature (e.g. based on a temperature sensor in the capsule), in a manner similar to that described above. The controller may be part of the control column, or may be part of another element of the surgical system, e.g. in the capsule 116.

The coolant conduit preferably has an undulating (e.g. serpentine) configuration and is formed of a thermally conductive material. This may allow the coolant fluid to draw more heat from the capsule 116 and/or from the control column itself (which may itself include a heat sink). In this embodiment, the coolant conduit (not shown) extends upwards from the inlet 306 toward the recess 126, and downwards again towards the outlet 308 (i.e. in a U-shape). This provides a flow path having closer thermal communication with the recess 126, so that the coolant fluid may more effectively draw heat from the capsule when in use.

In variant embodiments, which are not illustrated, the coolant conduit may instead circulate around the outer surface of the control column, which may then not include an inlet 306 or outlet 308.

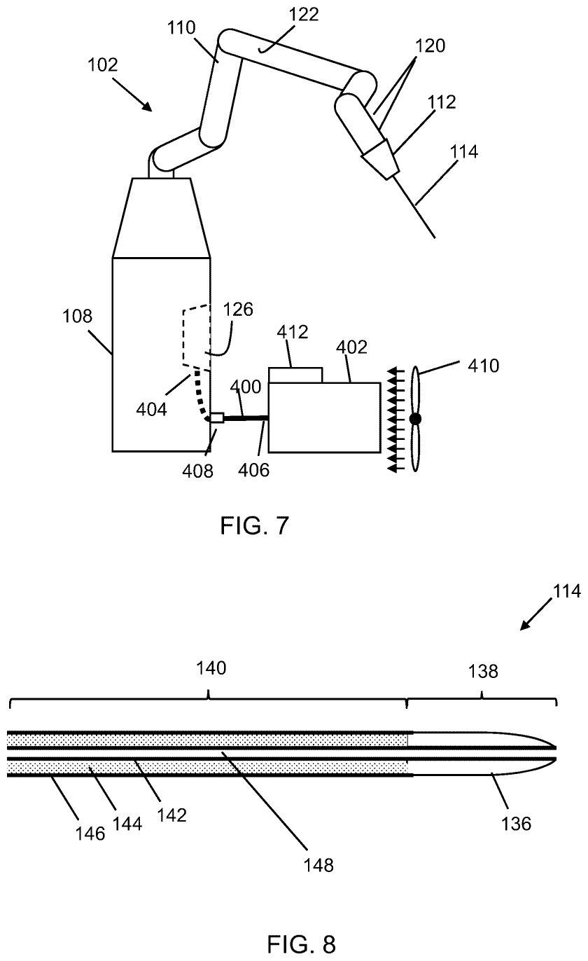

FIG. 7 is a schematic diagram of a robotic surgical tool 102 according to an embodiment of the invention.

The robotic surgical tool 102 of FIG. 7 is similar to that previously described with reference to FIG. 6, but is provided with a different cooling arrangement.

In FIG. 7, the cooling arrangement includes a heat pipe 400 to thermally connect the capsule 116 to a heat sink 402 when in use. The heat pipe extends from a first region 404 to a second region 406, and carries a fluid therebetween. In this embodiment, the first region 404 is located inside the control column 108 (as illustrated by dashed lines) and is located proximal to the recess 126, so as be in thermal communication with the capsule 116 in use. The second region 406 is mounted to the heat sink 402, which is located outside of the robotic surgical tool 102. An outlet 408 is provided in the wall of the control column 108 to allow the heat pipe 400 to extend therethrough, to fluidically connect the first region 404 and the second region 406.

A heat pipe is a conduit for carrying a fluid between two regions of different temperatures. The heat pipe utilises properties of fluid phase transition of the fluid to effectively transfer heat from its first region (i.e. near the electrosurgical generator unit) to its second region (i.e. away from the electrosurgical generator unit). In use, the first region 404 may have a relatively high temperature if the capsule 116 begins to overheat. The fluid contained within the heat pipe 400 may absorb the heat at the first region 404, causing the fluid to transition from a liquid into a vapour, which then travels along the heat pipe 400 to the second region 406. The lower temperatures at the second region 406 may then cause the vapour to transition back into a liquid, releasing its latent heat in the second region 406 for dissipation at the heat sink 402. The liquid then returns to the first region 404 (e.g. via a wick in the heat pipe 400) and the process repeats.

In this embodiment, the cooling arrangement further includes cooling mechanisms for maintaining a low temperature at the heat sink 402. This may be particularly useful in conjunction with the heat pipe, so as to help maintain low temperatures to cause a phase transition at the second region 406.

For example, the cooling arrangement includes a fan 410 positioned to cool the heat sink 402. Additionally, the heat sink 402 includes fins (not illustrated) disposed on its outer surface. The fins define channels oriented towards the fan, so that airflow from the fan may travel through the channels, more effectively cooling the heat sink 402.

The cooling arrangement further includes a thermoelectric heat pump 412 (e.g. a Peltier cooler) mounted on the heat sink 402 to remove heat from the heat sink 402.

FIG. 8 is a schematic cross-sectional view through an electrosurgical instrument 114 that can be handled by an articulated robotic arm in an embodiment of the invention. The electrosurgical instrument 114 may be connectable to the electrosurgery capsule 116 via the articulated robotic arm in a manner discussed above. The electrosurgical instrument 114 may be arranged or configured to deliver EM radiation from a distal instrument tip (or distal assembly) 136 in order to treat biological tissue located at a treatment site at or near to the distal assembly. The electrosurgical instrument 114 may be any device which in use is arranged to use EM energy (e.g. RF energy, microwave energy) for the treatment of biological tissue. The electrosurgical instrument 114 may use the EM energy for any or all of resection, coagulation and ablation. For example, the instrument 114 may be a resection device, a pair of microwave forceps, or a snare that radiates microwave energy and/or couples RF energy, and an argon beam coagulator.

The electrosurgical instrument 114 includes an instrument feed structure 140 for conveying EM radiation (e.g. an EM signal) to a distal end 138. In this example, the feed structure 140 is a coaxial transmission line formed from an inner conductor 142 that is separated from an outer conductor 146. The inner conductor 142 is hollow to define a passageway 148 for delivery of fluid.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. An apparatus for a robot-assisted surgical system, the apparatus comprising:
a robotic surgical tool comprising an articulated robot arm for supporting an electrosurgical instrument; and
a cooling assembly for removing heat from an electrosurgical generator unit mounted to the robotic surgical tool, the electrosurgical generator unit for generating an electrosurgical signal for use by the electrosurgical instrument.

2. The apparatus of claim 1, further comprising a connector for mounting the electrosurgical generator unit to the robotic surgical tool.

3. The apparatus of claim 1, further comprising the electrosurgical generator unit, said electrosurgical generator unit comprising:
a housing;
a signal generator contained within the housing, the signal generator being configured to generate the electrosurgical signal for use by the electrosurgical instrument; and
an energy delivery structure to couple the electrosurgical signal into the robotic surgical tool.

4. The apparatus of claim 1, wherein the cooling assembly comprises a heat sink arranged to be in thermal communication with the electrosurgical generator unit when the electrosurgical generator unit is mounted to the robotic surgical tool.

5. The apparatus of claim 4, wherein the cooling assembly further comprises a heat pipe for thermally coupling between the heat sink and the electrosurgical generator unit when the electrosurgical generator unit is mounted to the robotic surgical tool.

6. The apparatus of claim 1, wherein the cooling assembly comprises a resilient, thermally conductive connector for positioning between the robotic surgical tool and the electrosurgical generator unit to press against the robotic surgical tool and the electrosurgical generator unit when the electrosurgical generator unit is mounted to the robotic surgical tool.

7. The apparatus of claim 1, wherein the cooling assembly comprises one or more fins arranged to be in thermal communication with the electrosurgical generator unit when the electrosurgical generator unit is mounted to the robotic surgical tool.

8. The apparatus of claim 1, wherein the cooling assembly comprises an electrically controlled active cooling mechanism for actively cooling the apparatus.

9. The apparatus of claim 8, wherein the robotic surgical tool comprises an energy delivery structure to provide electrical power to the active cooling mechanism.

10. The apparatus of claim 8, further comprising:
a sensor for detecting a temperature of the apparatus; and
a controller to control the active cooling mechanism based on the detected temperature.

11. The apparatus of claim 8, wherein the active cooling mechanism comprises a fan to direct air flow over a surface of the apparatus.

12. The apparatus of claim 8, wherein the active cooling mechanism comprises a heat pump to remove heat from the apparatus.

13. The apparatus of claim 8, wherein the apparatus comprises one or more conduits arranged to be in thermal communication with the electrosurgical generator unit when the electrosurgical generator unit is mounted to the robotic surgical tool, and wherein the active cooling mechanism is configured to circulate a coolant fluid through the one or more conduits.

14. The apparatus of claim 13, wherein the robotic surgical tool comprises the one or more conduits.

15. The apparatus of claim 1, wherein the robotic surgical tool comprises the cooling assembly.

16. An electrosurgical generator unit for mounting to a robotic surgical tool, the electrosurgical generator unit comprising:
a housing;
a signal generator contained within the housing, the signal generator being configured to generate an electrosurgical signal for use by an electrosurgical instrument supported by the robotic surgical tool;
an energy delivery structure to couple the electrosurgical signal into the robotic surgical tool; and
a cooling assembly for removing heat from the electrosurgical generator unit.
